(19) United States
(12) Patent Application Publication
(10) Pub. No.: US 2010/0106173 A1
(43) Pub. Date: Apr. 29, 2010

(54) ULTRASONIC SURGICAL DEVICE

(76) Inventor: Hideto Yoshimine, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/256,837

(22) Filed: Oct. 23, 2008

(51) Int. Cl. A61B 17/32

(52) U.S. Cl. 606/169

(57) ABSTRACT

There is disclosed an ultrasonic surgical instrument which comprises a sheath having a distal end and a proximal end and an ultrasonic probe to be inserted into the sheath, the ultrasonic probe having an interpolating portion positioned in the sheath and a hook-like treatment portion arranged in the distal end of the interpolating portion so that the treatment portion protrudes from the sheath, wherein the treatment portion has an extending portion which extends obliquely forward and downward from the upper portion of the distal end of the interpolating portion, and has a recess formed under the extending portion.

### BACKGROUND OF THE INVENTION

The present invention relates to an ultrasonic surgical instrument which is used to perform an operation such as the incision of a living tissue by utilization of ultrasonic vibration.

As one example of a usual ultrasonic surgical instrument which is used to perform an operation such as the incision of a living tissue by utilization of an ultrasonic vibration, for example, an ultrasonic surgical instrument disclosed in Jpn. Pat. Appln. KOKAI Publication No. 2000-254136 (Patent Document 1) is generally known. In this ultrasonic surgical instrument, the proximal end of an elongate inserting portion is connected to an operating portion on a hand side. This operating portion is provided with an ultrasonic transducer which generates ultrasonic vibration. The distal end of the inserting portion is provided with a treatment portion for treating the living tissue.

The inserting portion has an elongate tubular sheath. Into the sheath, a rod-like vibration transmitting member (a probe) is inserted. The proximal end of the vibration transmitting member is connected to the ultrasonic transducer. Moreover, the ultrasonic vibration generated by the ultrasonic transducer is transmitted to the distal end of the probe. The probe distal end is provided with a hook-like treatment portion.

During the ultrasonic operation, the hook-like portion of the treatment portion at the distal end is hooked on the living tissue, and in a state where tension is imparted to the living tissue, ultrasonic vibration is transmitted to the distal end of the probe. In consequence, the living tissue is incised by the treatment portion of the distal end of the inserting portion, and a bleeding part is coagulated using frictional heat due to contact with the living tissue.

### BRIEF SUMMARY OF THE INVENTION

An ultrasonic surgical instrument of one configuration according to the present invention comprises a sheath having a distal end and a proximal end; and an ultrasonic probe to be inserted into the sheath, the ultrasonic probe having an interpolating portion positioned in the sheath and a hook-like treatment portion arranged in the distal end of the interpolating portion so that the treatment portion protrudes from the sheath, wherein the treatment portion has an extending portion which extends obliquely forward and downward from the upper portion of the distal end of the interpolating portion, and has a recess formed under the extending portion.

Preferably, the treatment portion has a distal end hook portion which warps upwards from the distal end of the extending portion.

Preferably, the treatment portion is provided with the extending portion and the distal end hook portion in a balanced state in which the barycentric position of the whole treatment portion substantially matches that of the whole ultrasonic probe.

Preferably, the treatment portion has an axially parallel portion extended substantially in parallel with the axial direction of the ultrasonic probe between the extending portion and the distal end hook portion.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a sectional view showing the overall schematic constitution of an ultrasonic surgical instrument of a first embodiment according to the present invention;

FIG. 2 is a perspective view showing the distal end of a probe unit of the ultrasonic surgical instrument of the first embodiment;

FIG. 3 is a side view showing the distal end of the probe unit of the ultrasonic surgical instrument of the first embodiment;

FIG. 4 is a perspective view showing a distal end treatment portion of a vibration transmitting member of the ultrasonic surgical instrument of the first embodiment;

FIG. 5 is a side view showing a distal end treatment portion of the vibration transmitting member of the ultrasonic surgical instrument of the first embodiment;

FIG. 6 is a characteristic diagram of ultrasonic vibration in a case where a probe distal end of the ultrasonic surgical instrument of the first embodiment is normal; and

FIG. 7 is a characteristic diagram of the ultrasonic vibration in a case where the probe distal end of the ultrasonic surgical instrument of the first embodiment is abnormal.

### DETAILED DESCRIPTION OF THE INVENTION

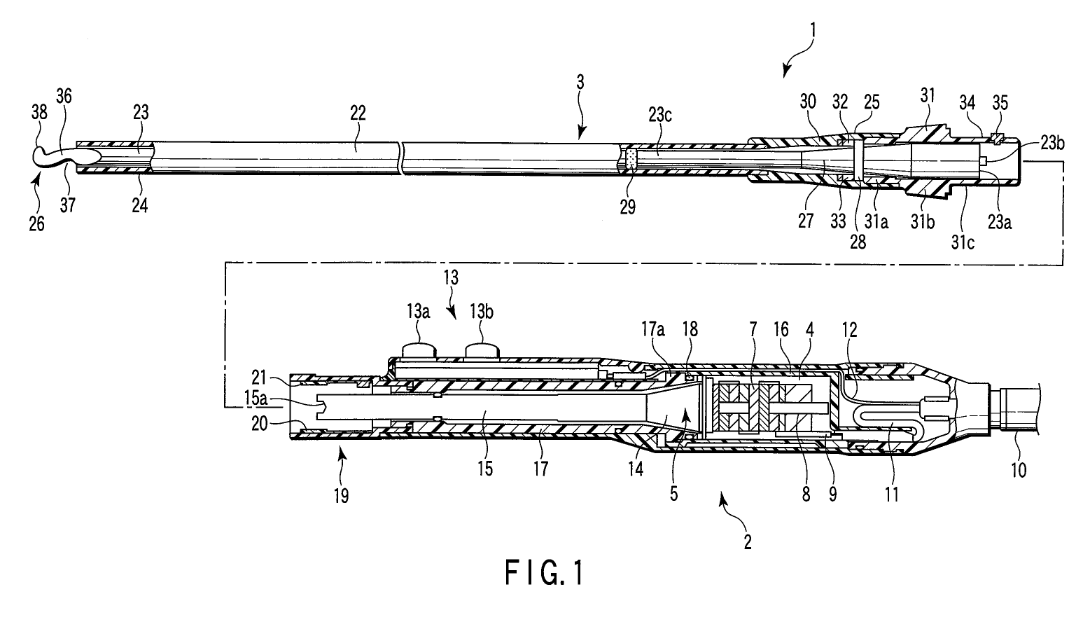

Hereinafter, a first embodiment of the present invention will be described with reference to FIGS. 1 to 7. FIG. 1 shows the overall schematic constitution of an ultrasonic surgical instrument 1 of the present embodiment. The ultrasonic surgical instrument 1 has a hand piece 2 and a probe unit 3.

The hand piece 2 has a casing 4 and a bolted Langevin type transducer (BLT) 5. The casing 4 is made of an electrically insulating resin material. The casing 4 contains the BLT 5 therein. The BLT 5 has a plurality of piezoelectric elements 7 and two electrodes 8, 9. The two electrodes 8, 9 come in contact with both end faces of the piezoelectric elements 7.

The proximal end of the hand piece 2 is connected to one end of a cord 10. The other end of this cord 10 is electrically connected to a driving power source (not shown). In the cord 10, an electric wire 11 for the transducer and an electric wire 12 for a switch are arranged. The electric wire 11 for the transducer in the cord 10 is connected to the two electrodes 8, 9, respectively. A driving power is supplied from the driving power source to the BLT 5 via the electric wire 11 for the transducer in the cord 10.

A switch 13 is disposed on the distal end side of the hand piece 2. The switch 13 has, for example, a first switch 13a for driving in a set output state, and a second switch 13b for driving in a maximum output state. The first switch 13a and the second switch 13b are electrically connected to a control circuit in the driving power source via the electric wire 12 for the switch. The first switch 13a makes it possible to drive with a set amplitude, and the second switch 13b makes it possible to drive with a maximum amplitude.

The front portion of the BLT 5 is connected to an output shaft portion 15 via a conical horn 14. The output shaft portion 15 of the BLT 5 is made of a titanium alloy. The distal end of the output shaft portion 15 extends to the vicinity of the distal end of the hand piece 2. The center of the distal end face of the output shaft portion 15 is provided with a recess 15a for connection.

As to the hand piece 2, a transducer cover 16 made of a resin and an output shaft portion cover 17 made of a resin are arranged in the casing 4. The transducer cover 16 is a cover member which covers the plurality of piezoelectric elements 7 of the BLT 5 and the two electrodes 8, 9. The front end of the transducer cover 16 is extended to the position of the horn 14.

The output shaft portion cover 17 is a tubular cover member which covers the output shaft portion 15 of the BLT 5. The proximal end of the output shaft portion cover 17 is provided with a circular connecting portion 17a having a diameter larger than that of another portion. The connecting portion 17a of the output shaft portion cover 17 is extended to a position where the connecting portion is inserted into the transducer cover 16. Moreover, the connecting portion 17a of the output shaft portion cover 17 is connected to the front end of the transducer cover 16 in a state in which the front end of the transducer cover 16 is fitted into the outer peripheral surface of the connecting portion 17a of the output shaft portion cover 17. A seal member such as an O-ring 18 is attached to a bonding face between the connecting portion 17a of the output shaft portion cover 17 and the front end of the transducer cover 16.

The distal end of the hand piece 2 is provided with a probe receiver 19. The probe receiver 19 is a cylindrical member formed of a resin material. The proximal end of the probe receiver 19 is connected in a state in which the proximal end is fitted into the outer peripheral surface of the distal end of the output shaft portion cover 17. The distal end of the probe receiver 19 is extended to a position where the distal end protrudes forwards from the distal end of the output shaft portion 15.

The inner peripheral surface of the probe receiver 19 is provided with a connecting portion 20 detachably connected to the probe unit 3. This connecting portion 20 is formed of, for example, a cam mechanism having a cam groove 21, a screw hole and the like.

The probe unit 3 has a sheath 22, and a vibration transmitting member (an ultrasonic probe) 23 arranged concentrically with this sheath 22. In the sheath 22, the proximal end of a tube 24 formed of an electrically insulating resin material is provided with a cylindrical grip portion 25 to be gripped by a user. The distal end of the grip portion 25 is connected in a state in which the distal end is fitted into the proximal end of the tube 24.

The vibration transmitting member 23 is made of a titanium alloy. The length of the vibration transmitting member 23 is set to an integral multiple of ½ of the wavelength of the driving frequency of the BLT 5. The distal end of the vibration transmitting member 23 is provided with a treatment portion 26, described later. The proximal end of the vibration transmitting member 23 is provided with a horn 27 having a conically tapered face. This horn 27 is capable of enlarging the amplitude of the treatment portion 26 to a necessary amplitude. Moreover, the middle portion of the horn 27 of the vibration transmitting member 23 is provided with a flange 28. The flange 28 is arranged in a vibration nodal position. The center of a rear end face 23a of the vibration transmitting member 23 is provided with a protruding portion 23b to be detachably engaged with the connecting recess 15a of the output shaft portion 15.

A plurality of annular lining rubbers 29 are attached to the portion of the vibration transmitting member 23 covered with the tube 24. The lining rubbers 29 are arranged in the vibration nodal positions. The vibration transmitting member 23 and the tube 24 are arranged around the same center via the lining rubbers 29 without being brought into contact with each other.

The grip portion 25 has a front member 30 and a rear member 31. The rear end of the front member 30 is provided with an engagement hole 32 into which the flange 28 is inserted. The inner diameter of this engagement hole 32 is substantially equal to the diameter of the flange 28.

The rear member 31 has three portions; 31a, 31b and 31c having different outer diameters. The three portions 31a, 31b and 31c are the front portion 31a arranged in the axial direction of the sheath 22 on a front side, the middle portion 31b arranged at the middle, and the rear portion 31c arranged on a rear side. The outer diameter of the front portion 31a is set to such a size that the front portion is inserted into the engagement hole 32 of the front member 30. The inner diameter of the front portion 31a is set to a diameter smaller than that of the flange 28. The diameter of the middle portion 31b is set to a diameter larger than the outer diameter of the rear end of the front member 30.

An annular pressing member 33 is arranged in the engagement hole 32 of the front member 30. Moreover, the front end of the flange 28 inserted into the engagement hole 32 of the front member 30 of the grip portion 25 is allowed to abut on the pressing member 33. In this state, the front portion 31a of the rear member 31 is inserted into the engagement hole 32 of the front member 30 to sandwich the flange 28 between the front portion 31a of the rear member 31 and the pressing member 33. In consequence, the sheath 22 and the vibration transmitting member 23 are fixed via the flange 28.

The rear portion 31c on the proximal end of the grip portion 25 is formed into such a size that the rear portion is detachably inserted into the probe receiver 19 of the distal end of the hand piece 2. The rear portion 31c is provided with a connecting portion 34 detachably connected to the probe receiver 19 of the distal end of the hand piece 2. This connecting portion 34 has, for example, an engaging claw 35 to be engaged with the cam groove 21 of the probe receiver 19 of the hand piece 2. When the hand piece 2 is connected to the probe unit 3, the engaging claw 35 of the probe unit 3 is engaged with the cam groove 21 of the probe receiver 19. At this time, the protruding portion 23b of the rear end face 23a of the vibration transmitting member 23 is inserted into the connecting recess 15a of the output shaft portion 15, and detachably engaged with the recess. In this state, the rear end face 23a of the vibration transmitting member 23 and the output end of the output shaft portion 15 of the BLT 5 are pressed on each other, so that the ultrasonic vibration can be transmitted from the BLT 5 to the vibration transmitting member 23. It is to be noted that when the connecting portion 20 of the probe unit 3 is a screw hole, the connecting portion 34 of the grip portion 25 is formed of an external thread to be engaged with the screw hole of the probe unit 3.

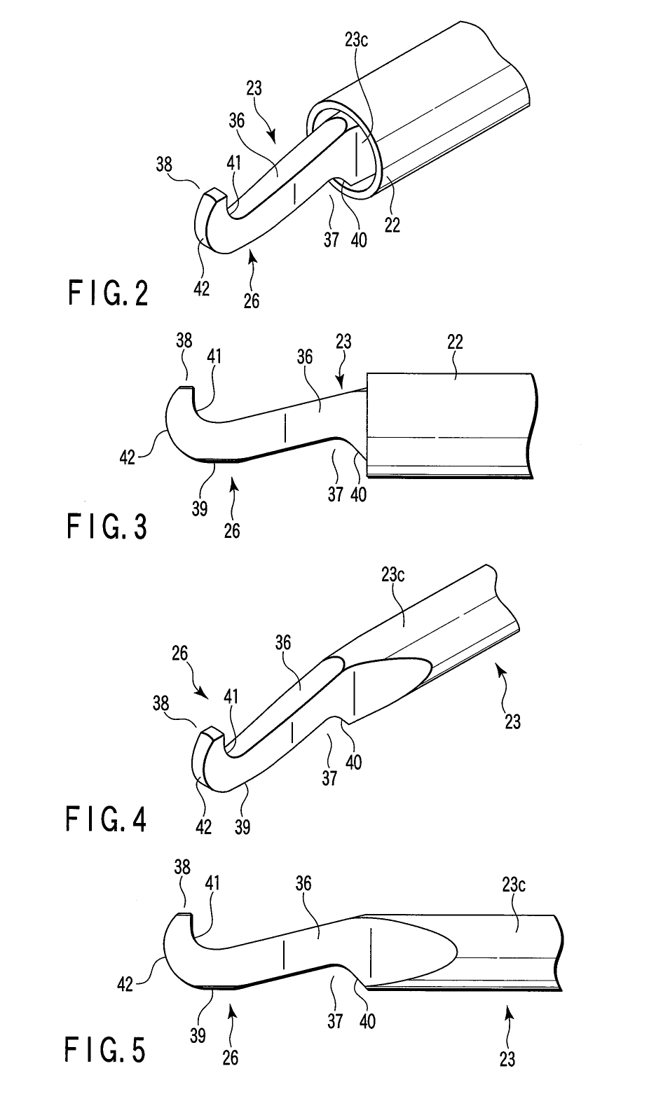

FIGS. 2 and 3 show the distal end of the probe unit 3. The vibration transmitting member 23 has an interpolating portion 23c positioned in the sheath 22, and the hook-like treatment portion 26 arranged on the distal end of the interpolating portion 23c so that the operating portion protrudes from the sheath 22. The interpolating portion 23c of the vibration transmitting member 23 is formed of a round rod having a substantially circular section. The treatment portion 26 is formed of a plate-like member obtained by processing both side faces of the round rod portion of the interpolating portion 23c so that they are flat and substantially in parallel with each other.

FIGS. 4 and 5 show the treatment portion 26 on the distal end of the vibration transmitting member 23. The treatment portion 26 has an extending portion 36 which extends obliquely forward and downward from the upper portion of the distal end of the interpolating portion 23c. A recess 37 is formed under the extending portion 36.

The treatment portion 26 has a distal end hook portion 38 which warps upwards from the distal end of the extending portion 36. Furthermore, the treatment portion 26 has an axially parallel portion 39 extended substantially in parallel with the axial direction of the vibration transmitting member 23 between the extending portion 36 and the distal end hook portion 38.

Moreover, the proximal end of the extending portion 36 is provided with a smoothly curved face (R-portion) 40 connected to the lower end portion of the distal end of the interpolating portion 23c of the vibration transmitting member 23. This curved face 40 forms the wall face of the recess 37. Furthermore, a portion connecting the axially parallel portion 39 to the distal end hook portion 38 is provided with upper and lower curved portions (an upper curved portion 41, a lower curved portion 42) which are smoothly curved faces.

Furthermore, the distal end hook portion 38 is arranged on the upper surface of the treatment portion 26, and the recess 37 is arranged on the lower surface of the operating portion. In consequence, the extending portion 36 and the distal end hook portion 38 are formed in a balanced state in which the barycentric position of the whole treatment portion 26 substantially matches that of the whole vibration transmitting member 23.

Next, an operation of the present embodiment having the above constitution will be described. To use the ultrasonic surgical instrument 1 of the present embodiment, the ultrasonic surgical instrument is set to a state in which the hand piece 2 is connected to the probe unit 3. At this time, the rear portion 31c of the grip portion 25 of the probe unit 3 is inserted into the probe receiver 19 on the distal end of the hand piece 2. Moreover, the engaging claw 35 of the probe unit 3 is engaged with the cam groove 21 of the probe receiver 19. At this time, the protruding portion 23b of the rear end face 23a of the vibration transmitting member 23 is inserted into the connecting recess 15a of the output shaft portion 15 and detachably engaged with the recess. In this state, the rear end face 23a of the vibration transmitting member 23 and the output end of the output shaft portion 15 of the BLT 5 are pressed on each other, and assembled in a state in which the ultrasonic vibration can be transmitted from the BLT 5 to the vibration transmitting member 23.

To perform an ultrasonic operation, the distal end hook portion 38 of the treatment portion 26 on the distal end of the probe unit 3 is hooked on a living tissue. Moreover, the ultrasonic vibration is transmitted to the treatment portion 26 on the distal end of the probe unit 3 in a state in which tension is imparted to the living tissue. In consequence, the living tissue is incised by the treatment portion 26 on the distal end of the probe unit 3, and a bleeding part is allowed coagulate by frictional heat due to contact with the living tissue.

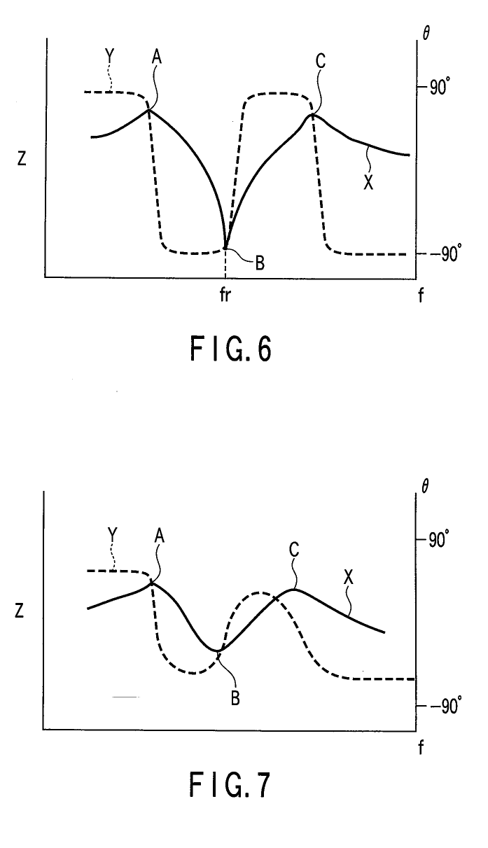

Furthermore, in the present embodiment, a failure detection system having a constitution in which the change of a vibration system for performing the ultrasonic vibration is detected by the driving power source to detect the failure of the ultrasonic surgical instrument 1 during the ultrasonic operation is incorporated in the driving power source. FIG. 6 is a characteristic diagram of ultrasonic vibration in a case where the treatment portion 26 on the distal end of the probe unit 3 of the ultrasonic surgical instrument 1 is normal. In FIG. 6, the abscissa indicates a frequency (f), the ordinate indicates an impedance (z), a solid-line characteristic curve X indicates the characteristic curve of the ultrasonic vibration, and a dotted-line characteristic curve Y indicates a phase (θ), respectively. Moreover, point B is an impedance resonance point, and points A and C are antiresonance points. Usually, at the resonance point B, the driving power source is controlled into a state in which the treatment portion 26 on the distal end of the probe unit 3 is driven.

Moreover, in the present embodiment, the ultrasonic operation is performed in a state in which the distal end hook portion 38 of the probe unit 3 is hooked on the living tissue. At this time, the treatment portion 26 has the extending portion 36 which extends obliquely forward and downward from the upper portion of the distal end of the interpolating portion 23c. Therefore, when the distal end hook portion 38 of the probe unit 3 is hooked on the living tissue, a maximum stress is generated in the curved face 40 of the proximal end of the extending portion 36 in the treatment portion 26. In this state, when a crack or the like is generated in the treatment portion 26 during an ultrasonic operation, a crack or the like is generated in the curved face 40 of the proximal end of the extending portion 36.

Therefore, in a case where a crack or the like is generated in the treatment portion 26 during an ultrasonic operation, the volume of the distal end of the operating portion from the portion of the curved face 40 in which the crack is generated increases as compared with a case where the crack is generated in the upper curved portion 41. Consequently, the change in the vibration system (e.g., a resonance frequency (f), an impedance (z), a phase (θ) or the like shown in FIG. 7) in a case where the crack or the like is generated in the treatment portion 26 increases. In this case, the change in the vibration system for performing the ultrasonic vibration is easily detected by the driving power source during the ultrasonic operation, so that the failure of the treatment portion 26 is easily detected during the ultrasonic operation.

The present embodiment produces an effect as follows. That is, in the ultrasonic surgical instrument 1 of the present embodiment, the treatment portion 26 is formed so as to include the extending portion 36 which extends obliquely forward and downward from the upper portion of the distal end of the interpolating portion 23c. Consequently, in a case where the distal end hook portion 38 of the probe unit 3 is hooked on the living tissue, the portion of the treatment portion 26 in which the maximum stress is generated can be set to the curved face 40 of the proximal end of the extending portion 36. Therefore, in a case where a crack or the like is generated in the treatment portion 26 during the ultrasonic operation, adjustment can be performed so that a crack or the like is generated in the curved face 40 of the proximal end of the extending portion 36 in which the maximum stress is generated. Consequently, the characteristic change of the vibration system for performing the ultrasonic vibration in a case where a crack or the like is generated in the treatment portion 26 can be increased during the ultrasonic operation, so that any change in the vibration system for performing the ultrasonic vibration can easily be detected by the driving power source during the ultrasonic transducer. Therefore, a state in which a crack or the like is generated in the treatment portion 26 can quickly be detected during an ultrasonic operation, so that even when the size of the treatment portion 26 is decreased for a delicate operation, the driving power source can be stopped before the generation of severe damage such as breakage of the treatment portion 26, to prevent such damage from occurring.

Moreover, in the present embodiment, the treatment portion 26 has the extending portion 36 which extends obliquely forward and downward from the upper portion of the distal end of the interpolating portion 23c, and the recess 37 is formed under the extending portion 36. In consequence, since the recess 37 is provided, the treatment portion 26 can be lightened, so that a moment generated in the treatment portion 26 during the ultrasonic vibration can be decreased. Consequently, a crack or the like is not easily generated in the treatment portion 26 during an ultrasonic operation, which improves the durability.

Furthermore, the distal end hook portion 38 is arranged on the upper surface of the treatment portion 26, and the recess 37 is arranged on the lower surface, so that the operating portion is formed in a balanced state in which the barycentric position of the whole treatment portion 26 substantially matches that of the whole vibration transmitting member 23. In consequence, there is an effect that lateral vibration transmitted via the vibration transmitting member 23 can be suppressed.

It is to be noted that the present invention is not limited to the above embodiment, and needless to say, the present invention can variously be modified without departing from the scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An ultrasonic surgical instrument comprising:
a sheath having a distal end and a proximal end; and
an ultrasonic probe to be inserted into the sheath, the ultrasonic probe having an interpolating portion positioned in the sheath and a hook-like treatment portion arranged in the distal end of the interpolating portion so that the treatment portion protrudes from the sheath,
wherein the treatment portion has an extending portion which extends obliquely forward and downward from the upper portion of the distal end of the interpolating portion, and has a recess formed under the extending portion.

2. The ultrasonic surgical instrument according to claim 1, wherein the treatment portion has a distal end hook portion which warps upwards from the distal end of the extending portion.

3. The ultrasonic surgical instrument according to claim 2, wherein a barycentric position of the treatment portion is located on the longitudinal axis of the ultrasonic probe.

4. The ultrasonic surgical instrument according to claim 2, wherein the treatment portion has an axially parallel portion extended substantially in parallel with the axial direction of the ultrasonic probe between the extending portion and the distal end hook portion.
